(19) United States
(12) Patent Application Publication
(10) Pub. No.: US 2017/0303987 A1
(43) Pub. Date: Oct. 26, 2017

(54) ULTRASONIC TREATMENT APPARATUS

(71) Applicant: OLYMPUS CORPORATION, Tokyo (JP)

(72) Inventors: Kei HORIE, Tokyo (JP); Miyuki MURAKAMI, Tokyo (JP)

(73) Assignee: OLYMPUS CORPORATION, Tokyo (JP)

(21) Appl. No.: 15/647,695

(22) Filed: Jul. 12, 2017

Related U.S. Application Data
(63) Continuation of application No. PCT/JP2015/051669, filed on Jan. 22, 2015.

(51) Int. Cl. A61B 18/12; A61B 18/04; A61B 18/00

(52) U.S. Cl. CPC A61B 18/12; A61B 18/04; A61B 2018/00041

(57) ABSTRACT

Provided is an ultrasonic treatment apparatus provided with: a treatment-ultrasonic-wave irradiator that irradiates the biological tissue with focused ultrasonic waves, thus heating the vicinity of a focal point of the focused ultrasonic waves at a deep portion of the biological tissue to a temperature that is equal to or greater than a thermal-denaturation temperature of the biological tissue; and a pre-heating-energy irradiator that irradiates the biological tissue with energy waves, thus heating the vicinity of the focal point to a temperature that is less than the thermal-denaturation temperature, wherein the pre-heating-energy irradiator irradiates the biological tissue with the energy waves from a direction different from the direction in which the treatment-ultrasonic-wave irradiator irradiates with the focused ultrasonic waves.

### CROSS-REFERENCE TO RELATED APPLICATIONS

This is a continuation of International Application PCT/JP2015/051669, with an international filing date of Jan. 22, 2015, which is hereby incorporated by reference herein in its entirety. This application claims the benefit of International Application PCT/JP2015/051669, the content of which is incorporated herein by reference.

### BACKGROUND OF THE INVENTION

### Field of the Invention

The present invention relates to an ultrasonic treatment apparatus.

### Description of the Related Art

In the related art, in treatment of biological tissue by means of ultrasonic waves (focused ultrasonic waves) that are focused on one point, an ultrasonic treatment apparatus has been proposed that has a pre-heating mode and an ablation mode for heating a region corresponding to an affected portion in the biological tissue and that heats the biological tissue in two steps (for example, see Japanese Unexamined Patent Application, Publication No. 2000-175933). In Japanese Unexamined Patent Application, Publication No. 2000-175933, first, in the pre-heating mode, the biological tissue is irradiated with weak ultrasonic waves to preheat the biological tissue to a temperature that is less than a thermal-denaturation temperature. Subsequently, in the ablation mode, the pre-heated biological tissue is irradiated with ultrasonic waves to heat the biological tissue to a temperature that is equal to or greater than the thermal-denaturation temperature, thus ablating the biological tissue. By doing so, in the ablation mode, it is possible to ablate the biological tissue in a short period of time by using weak ultrasonic waves.

### SUMMARY OF INVENTION

An aspect of the present invention provides an ultrasonic treatment apparatus comprising: a treatment-ultrasonic-wave irradiator that is disposed facing a surface of a biological tissue and that is configured to irradiate the biological tissue with focused ultrasonic waves, thus heating a vicinity of a focal point of the focused ultrasonic waves positioned at a deep portion in the biological tissue to a temperature that is equal to or greater than a thermal-denaturation temperature of the biological tissue; and a pre-heating-energy irradiator that is configured to irradiate the biological tissue with energy waves, thus heating the vicinity of the focal point to a temperature that is less than the thermal-denaturation temperature, wherein the pre-heating-energy irradiator is configured to irradiate the biological tissue with the energy waves from a direction that is different from the direction in which the treatment-ultrasonic-wave irradiator irradiates with the focused ultrasonic waves.

### BRIEF DESCRIPTION OF DRAWINGS

FIG. 1 is a block diagram showing the overall configuration of an ultrasonic treatment apparatus according to an embodiment of the present invention.

FIG. 2 is a diagram showing the configuration of a distal-end portion of an inserted portion of the ultrasonic treatment apparatus in FIG. 1.

FIG. 3 is a diagram showing a modification of a pre-heating ultrasonic-wave irradiating portion of the ultrasonic treatment apparatus in FIG. 1.

FIG. 4 is a diagram showing a modification of a treatment-ultrasonic-wave irradiating portion of the ultrasonic treatment apparatus in FIG. 1.

FIG. 5 is an overall configuration diagram showing a modification of the ultrasonic treatment apparatus in FIG. 1.

FIG. 6 is an overall configuration diagram showing another modification of the ultrasonic treatment apparatus in FIG. 1.

FIG. 7 is a diagram for explaining examples of pre-heating operation and ablating operation of the ultrasonic treatment apparatus in FIG. 6.

FIG. 8 is a diagram showing a region to be pre-heated and ablated in the pre-heating operation and the ablating operation in FIG. 7.

FIG. 9 is a diagram for explaining other examples of the pre-heating operation and ablating operation of the ultrasonic treatment apparatus in FIG. 6.

FIG. 10 is a diagram showing a region to be pre-heated and ablated in the pre-heating operation and the ablating operation in FIG. 9.

FIG. 11 is a diagram for explaining a method of adjusting the intensity of treatment ultrasonic waves in the ablating operation in FIGS. 7 and 9.

FIG. 12 is a diagram for explaining another modification of the ultrasonic treatment apparatus in FIG. 1 and an example of a method of using the same.

FIG. 13 is a diagram for explaining another modification of the ultrasonic treatment apparatus in FIG. 1 and an example of a method of using the same.

FIG. 14 is a diagram for explaining another modification of the ultrasonic treatment apparatus in FIG. 1 and an example of a method of using the same.

FIG. 15 is a diagram showing a modification of the ultrasonic treatment apparatus in FIG. 14.

FIG. 16 is a diagram showing another modification of the ultrasonic treatment apparatus in FIG. 14.

FIG. 17 is a diagram showing another modification of the ultrasonic treatment apparatus in FIG. 14.

FIG. 18A is a diagram showing another modification of the ultrasonic treatment apparatus in FIG. 14.

FIG. 18B is a diagram in which a treatment-ultrasonic-wave irradiating portion and a microwave irradiating portion of the ultrasonic treatment apparatus in FIG. 18A are viewed from the front.

FIG. 19 is a diagram showing another modification of the ultrasonic treatment apparatus in FIG. 1.

FIG. 20 is a diagram showing a modification of the ultrasonic treatment apparatus in FIG. 19.

FIG. 21 is an overall configuration diagram showing another modification of the ultrasonic treatment apparatus in FIG. 1.

### DESCRIPTION OF EMBODIMENT

An ultrasonic treatment apparatus 1 according to an embodiment of the present invention will be described below with reference to the drawings.

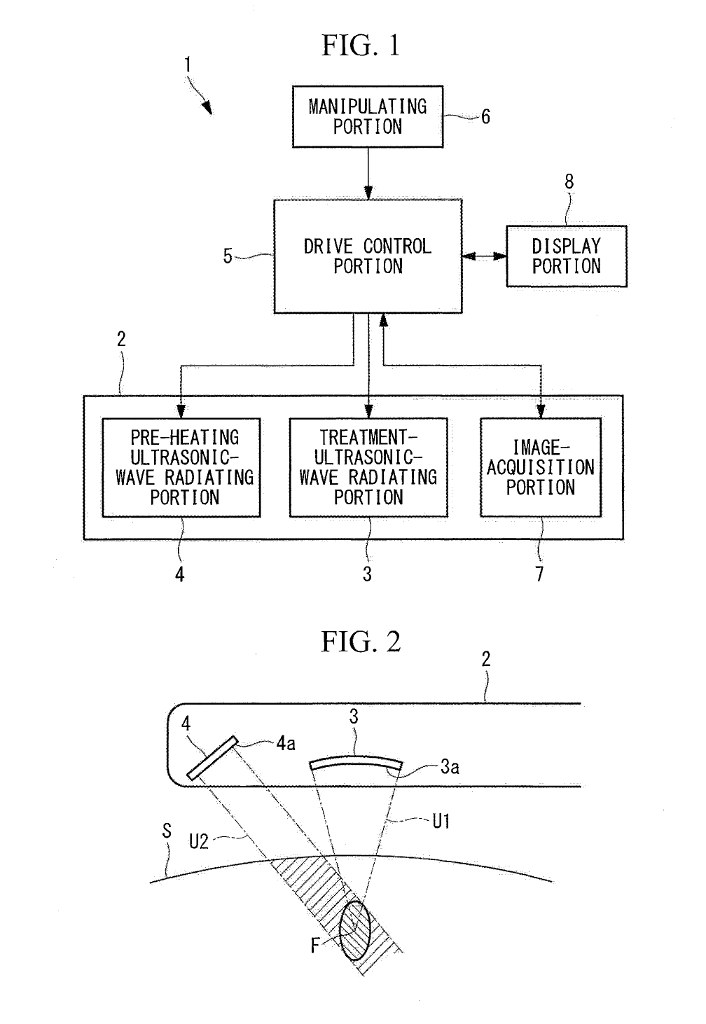

As shown in FIGS. 1 and 2, the ultrasonic treatment apparatus 1 according to this embodiment is provided with: a treatment-ultrasonic-wave irradiating portion (treatment-ultrasonic-wave irradiator) 3 and a pre-heating ultrasonic-wave irradiating portion (pre-heating-energy irradiator) 4 that are provided at a distal-end portion of an elongated inserted portion 2 that can be inserted into a living subject; a drive control portion 5 (controller) that controls driving of the two ultrasonic-wave irradiating portions 3 and 4; a manipulating portion 6 with which a user manipulates the operation of the ultrasonic-wave irradiating portions 3 and 4; an image-acquisition portion 7 that acquires ultrasonic-wave images of biological tissue S; and a display portion 8 that displays the ultrasonic-wave images.

The treatment-ultrasonic-wave irradiating portion 3 is provided with, for example, an ultrasonic-wave transducer, such as an HIFU (High Intensity Focused Ultrasound) element, having a concave emitting surface 3a, and emits, from the emitting surface 3a, treatment ultrasonic waves U1 that are focused at a focal point F of the emitting surface 3a when driving signals are provided to the HIFU element from the drive control portion 5. As shown in FIG. 2, when the biological tissue S is irradiated with the treatment ultrasonic waves U1 in a state in which the focal point F is positioned at a deep portion of the biological tissue S, the temperature at the focal point F increases most rapidly, and, additionally, a three-dimensional region centered on the focal point F is heated due to the propagation of heat from the focal point F to the surrounding area thereof. The heating region centered on the focal point F inside the biological tissue S is a nearly elliptical area that has the long axis along the center axis of the irradiated beam. The shape of the emitting surface 3a of the treatment-ultrasonic-wave irradiating portion 3 need not have a concave shape so long as it has a shape with which a focal point can be formed.

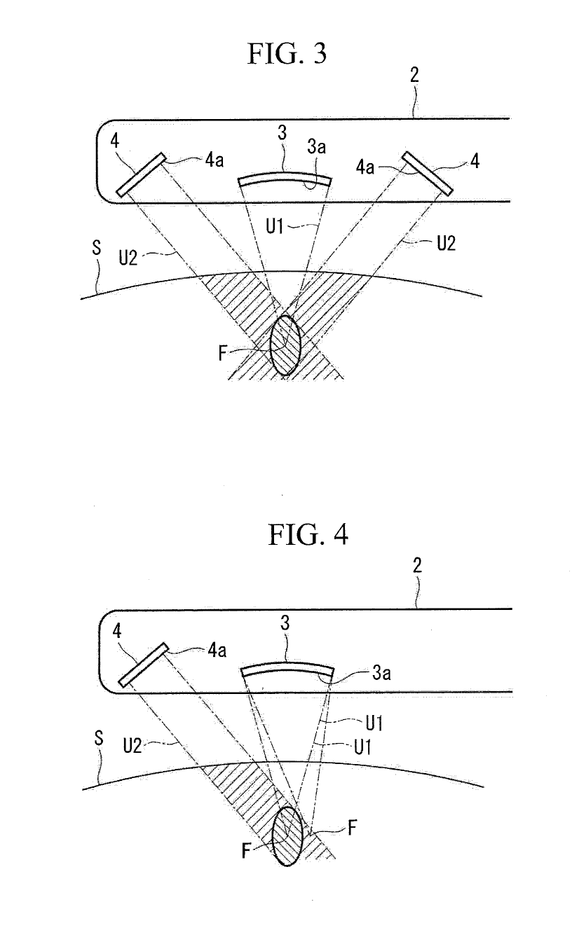

The pre-heating ultrasonic-wave irradiating portion 4 is provided with an ultrasonic wave element having a flat emitting surface 4a, and emits, from the emitting surface 4a, pre-heating ultrasonic waves (pre-heating energy waves) U2 when driving signals are provided to the ultrasonic wave element from the drive control portion 5. When the biological tissue S is irradiated with the pre-heating ultrasonic waves U2, the temperature in the irradiation region of the pre-heating ultrasonic waves U2 is evenly increased. As shown in FIG. 3, multiple units of the pre-heating ultrasonic-wave irradiating portion 4 may be provided. In addition, the emitting surface 4a has a curvature with which nearly parallel irradiation pathways are formed in order to achieve a pre-heating effect near the affected portion, and, by doing so, it is possible to effectively heat a large pre-heating region. Meanwhile, it is also possible to pre-heat a large region by performing pre-heating at a plurality of focal-point positions F. Furthermore, as with the emitting surface 3a of the treatment-ultrasonic-wave irradiating portion, the emitting surface 4a may be concavely shaped, and the pre-heating ultrasonic waves U2 may irradiate so as to heat a region to be pre-heated in the surroundings thereof to cause thermal diffusion.

The treatment-ultrasonic-wave irradiating portion 3 and the pre-heating ultrasonic-wave irradiating portion 4 are disposed in a manner in which the emitting surfaces 3a and 4a are inclined with respect to each other so that the sound axis of the treatment ultrasonic waves U1 and the sound axis of the pre-heating ultrasonic waves U2 intersect with each other at the focal point F. By doing so, although the treatment ultrasonic waves U1 and the pre-heating ultrasonic waves U2 overlap with each other at the focal point F, in portions between the emitting surfaces 3a and 4a and the focal point F, the treatment ultrasonic waves U1 and the pre-heating ultrasonic waves U2 are propagated in separate pathways without overlapping with each other except for the heating region to be treated. Therefore, the surface of and inside the biological tissue S are not heated by the pre-heating ultrasonic waves U2 in the portions between the emitting surface 3a and the focal point F.

Here, the pre-heating ultrasonic waves U2 possess energy with which it is possible to heat the biological tissue S to a temperature (for example, about 50° C.) that is less than a thermal-denaturation temperature at which the biological tissue S is thermally-denatured. The treatment ultrasonic waves U1 possess energy with which it is possible to heat, in the vicinity of the focal point F thereof, the biological tissue S is pre-heated by the pre-heating ultrasonic waves U2 to a temperature (for example, about 70° C.) that is equal to or greater than the thermal-denaturation temperature.

As shown in FIG. 4, the treatment-ultrasonic-wave irradiating portion 3 may be configured so that the position of the focal point F in the irradiation area of the pre-heating ultrasonic waves U2 can be moved.

The drive control portion 5 executes a pre-heating operation in which the biological tissue S is heated by the pre-heating ultrasonic waves U2 by activating the pre-heating ultrasonic-wave irradiating portion 4 for a predetermined time, and, subsequently, executes an ablating operation in which the vicinity of the focal point F is additionally heated by the treatment ultrasonic waves U1 by activating the treatment-ultrasonic-wave irradiating portion 3. By doing so, the biological tissue S is first pre-heated, in the irradiation region of the pre-heating ultrasonic waves U2 including the focal point F, to a temperature that is greater than the body temperature and less than the thermal-denaturation temperature, and is subsequently ablated by being heated, only in the vicinity of the focal point F in the pre-heated region, to a temperature that is equal to or greater than the thermal-denaturation temperature.

The manipulating portion 6 is configured so as to allow a user to input start instructions and stop instructions for the treatment performed by the ultrasonic-wave irradiating portions 3 and 4. In addition, the manipulating portion 6 is configured so as to allow the user to input the irradiation conditions of the respective ultrasonic waves U1 and U2 (for example, frequencies and intensities of the respective ultrasonic waves U1 and U2, and irradiation time of the pre-heating ultrasonic waves U2 in the pre-heating operation). Instead of having the user input the individual instructions and conditions via the manipulating portion 6, the operation thereof may be automated so that the drive control portion 5 executes control for driving of the ultrasonic-wave irradiating portions 3 and 4 on the basis of conditions that are set in advance.

The image-acquisition portion 7 is provided with an ultrasonic-wave probe (not shown) that is provided in the vicinity of the ultrasonic-wave irradiating portions 3 and 4 and transmits and receives diagnostic ultrasonic waves to and from an area including the focal point F. The image-acquisition portion 7 generates an ultrasonic-wave image of the biological tissue S on the basis of ultrasonic-wave information received by means of the ultrasonic-wave probe and outputs the generated ultrasonic-wave image to the display portion 8.

Note that, it suffices that the image-acquisition portion 7 be a means with which it is possible to ascertain the relative positions between the treatment-ultrasonic-wave irradiating portion 3 and the biological tissue S, and the image-acquisition portion 7 may be, for example, an external imaging apparatus such as an MRI (magnetic resonance imaging) apparatus or the like.

Next, the operation of the thus-configured ultrasonic treatment apparatus 1 according to this embodiment will be described.

In order to treat an affected portion that is positioned in a deep portion of the biological tissue S by using the ultrasonic treatment apparatus 1 according to this embodiment, the treatment-ultrasonic-wave irradiating portion 3 is placed in a manner in which the emitting surface 3a faces a surface of the biological tissue S so that the focal point F of the treatment ultrasonic waves U1 is aligned with the affected portion. The positioning of the treatment-ultrasonic-wave irradiating portion 3 with respect to the affected portion is performed while checking the ultrasonic-wave image displayed on the display portion 8.

Next, on the basis of the start instruction for the treatment input via the manipulating portion 6, the drive control portion 5 starts to drive the treatment-ultrasonic-wave irradiating portion 3 and the pre-heating ultrasonic-wave irradiating portion 4, and the pre-heating operation and the ablating operation are sequentially executed. First, the drive control portion 5 activates the pre-heating ultrasonic-wave irradiating portion 4, thus the affected portion in the biological tissue S is irradiated with the pre-heating ultrasonic waves U2 for a predetermined time. By doing so, the affected portion is pre-heated to a temperature that is less than the thermal-denaturation temperature. Next, the drive control portion 5 activates the treatment-ultrasonic-wave irradiating portion 3, thus the affected portion is irradiated with the treatment ultrasonic waves U1. By doing so, the affected portion is heated to a temperature that is equal to or greater than the thermal-denaturation temperature. The user determines whether or not that affected portion has been ablated on the basis of the ultrasonic-wave image, and, when he/she determines that the affected portion has been ablated, he/she inputs the stop instruction for the treatment via the manipulating portion 6, thus stopping the irradiation of the treatment ultrasonic waves U1.

In this case, with this embodiment, the intensity and the irradiation time of the treatment ultrasonic waves U1 that are necessary to heat the region that has been pre-heated by the pre-heating ultrasonic waves U2 to a temperature that is equal to or greater than the thermal-denaturation temperature are weaker and shorter as compared with the intensity and the irradiation time that are necessary to heat the biological tissue S to a temperature that is equal to or greater than the thermal-denaturation temperature by using only the treatment ultrasonic waves U1. In other words, there is an advantage in that it is possible to ablate the affected portion by irradiating with relatively low-intensity treatment ultrasonic waves U1 for a short period of time.

In addition, because the inserted portion 2 of the internal ultrasonic treatment apparatus 1 has a small diameter and the sizes of the ultrasonic wave elements of the ultrasonic-wave irradiating portions 3 and 4 are limited to small sizes, the focal distance of the treatment ultrasonic waves U1 is small. Therefore, the distances from the emitting surfaces 3a and 4a to the biological tissue S are small, and the surface of the biological tissue S is also heated by the ultrasonic waves U1 and U2. With this embodiment, in regions other than the vicinity of the focal point F, only one of the pre-heating ultrasonic waves U2 and the treatment ultrasonic waves U1 are irradiated. Therefore, when the biological tissue S is irradiated with the treatment ultrasonic waves U1 until the affected portion is ablated, the regions other than the affected portion are not heated to a temperature that is equal to or greater than the thermal-denaturation temperature, and thus, there is an advantage in that it is possible to selectively ablate only the affected portion.

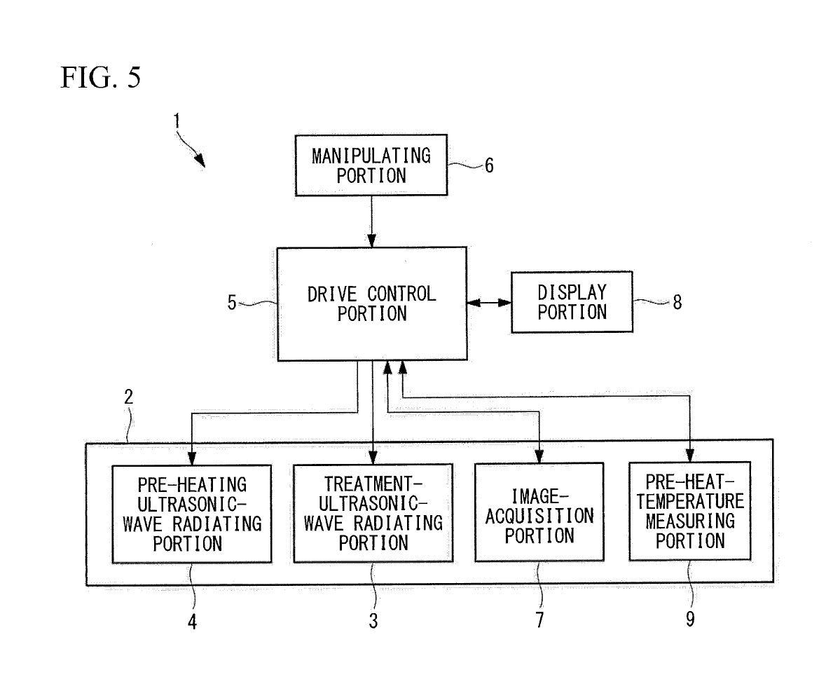

Note that, as shown in FIG. 5, this embodiment may be provided with a pre-heat-temperature measuring portion 9 (pre-heat-temperature measuring instrument) that measures the temperature in the vicinity of the focal point F that has been pre-heated in the pre-heating operation, and the drive control portion (treatment-ultrasonic-wave setting portion) 5 may set, on the basis of the temperature measured by the pre-heat-temperature measuring portion 9, the irradiation conditions of the treatment ultrasonic waves U1 by the treatment-ultrasonic-wave irradiating portion 3.

The pre-heat-temperature measuring portion 9 is provided with a temperature sensor that takes an actual measurement of the temperature in the vicinity of the focal point F. It is preferable that the temperature sensor be one that measures the temperature by using a non-contact method, for example, an infrared temperature sensor. In particular, in the case in which the affected portion is positioned in a deep portion, it is permissible to employ, as the pre-heat-temperature measuring portion 9, an apparatus that monitors the temperature of the affected portion by means of, for example, an MRI, or an apparatus that employs a method of estimating the temperature in the vicinity of the focal point F by measuring the surface temperature of the biological tissue S.

The drive control portion 5 has a function or a table in which the temperature in the vicinity of the focal point F and the irradiation conditions of the treatment ultrasonic waves U1 are associated with each other. The irradiation conditions are, for example, the intensity and the irradiation time of the treatment ultrasonic waves U1. In the function or the table, the temperature and the irradiation conditions are associated with each other so that the intensity or/and the irradiation time of the treatment ultrasonic waves U1 is decreased with an increase in the temperature in the vicinity of the focal point F. Subsequent to the pre-heating operation, the drive control portion 5 acquires the irradiation conditions of the treatment ultrasonic waves U1 associated with the temperature measured by the pre-heat-temperature measuring portion 9 from the function or the table, and the affected portion is irradiated with the treatment ultrasonic waves U1 in accordance with the acquired irradiation conditions.

The temperature of the pre-heating performed by the pre-heating ultrasonic waves U2 differs in accordance with the type of the biological tissue S, the environment, and so forth. Therefore, by measuring the temperature in the vicinity of the focal point F by using the pre-heat-temperature measuring portion 9 and by setting the irradiation conditions of the treatment ultrasonic waves U1 in accordance with the measured temperature, it is possible to reliably ablate the affected portion by the affected portion is adequately irradiated with the right amount of the treatment ultrasonic waves U1.

Instead of taking the actual measurement of the temperature in the vicinity of the focal point F by using the temperature sensor, the pre-heat-temperature measuring portion 9 may theoretically calculate the temperature in the vicinity of the focal point F on the basis of the irradiation conditions (for example, the intensity and the irradiation time) of the pre-heating ultrasonic waves U2 acquired from the drive control portion 5. In this case, the pre-heat-temperature measuring portion 9 calculates the temperature in the vicinity of the focal point F by using a function obtained on the basis of correlation between the irradiation conditions of the pre-heating ultrasonic waves U2 and the temperature in the vicinity of the focal point F, that is acquired, for example, by performing a preliminary experiment. In this case, because the temperature sensor is not necessary, it is possible to reduce the size of the apparatus.

The actual measured value or the calculated value of the temperature obtained by the pre-heat-temperature measuring portion 9 may be displayed on the display portion 8 in real-time so that the user can recognize the current temperature at the focal point F. By doing so, the user can effectively give, in the form of inputs via the manipulating portion 6, the start instruction and the stop instruction for the treatment performed by using the ultrasonic-wave irradiating portions 3 and 4. Furthermore, the operation may be automated so that the drive control portion 5 gives, on the basis of the actual measured value or the calculated value of the temperature obtained by the pre-heat-temperature measuring portion 9, the start instruction and the stop instruction of the treatment performed by the ultrasonic-wave irradiating portions 3 and 4.

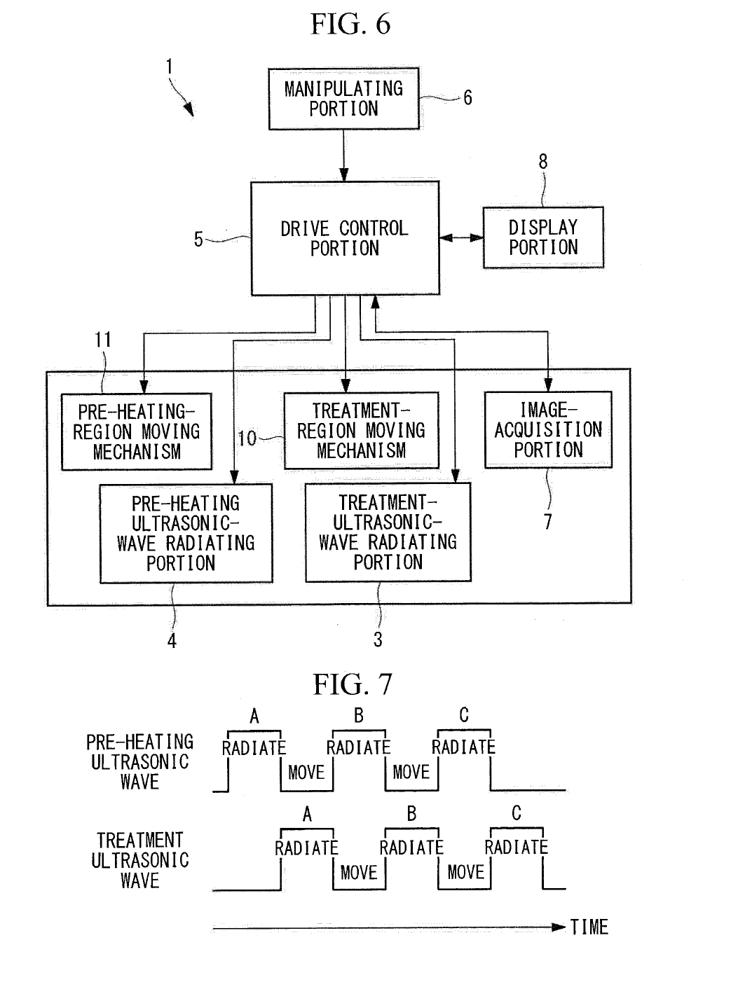

In addition, as shown in FIG. 6, this embodiment may be provided with a treatment-region moving mechanism 10 that moves the focal point F of the treatment ultrasonic waves U1 and a pre-heating-region moving mechanism 11 that moves the irradiation region of the pre-heating ultrasonic waves U2. In this case, as shown in FIGS. 7 to 10, the drive control portion (controller) 5 controls the pre-heating ultrasonic-wave irradiating portion 4 and the pre-heating-region moving mechanism 11 so that the radiation of the pre-heating ultrasonic waves U2 onto the biological tissue S and movement of the irradiation region of the pre-heating ultrasonic waves U2 are repeated in an alternating manner. In addition, the drive control portion 5 controls the treatment-ultrasonic-wave irradiating portion 3 and the treatment-region moving mechanism 10 so that the movement of the focal point F to a region that has been pre-heated by the pre-heating ultrasonic waves U2 in an immediately preceding step and the radiation of the treatment ultrasonic waves U1 onto the focal point F are repeated in an alternating manner.

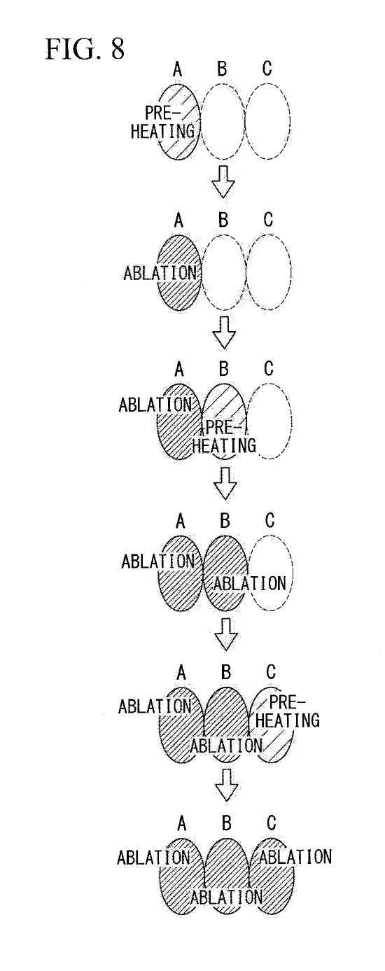

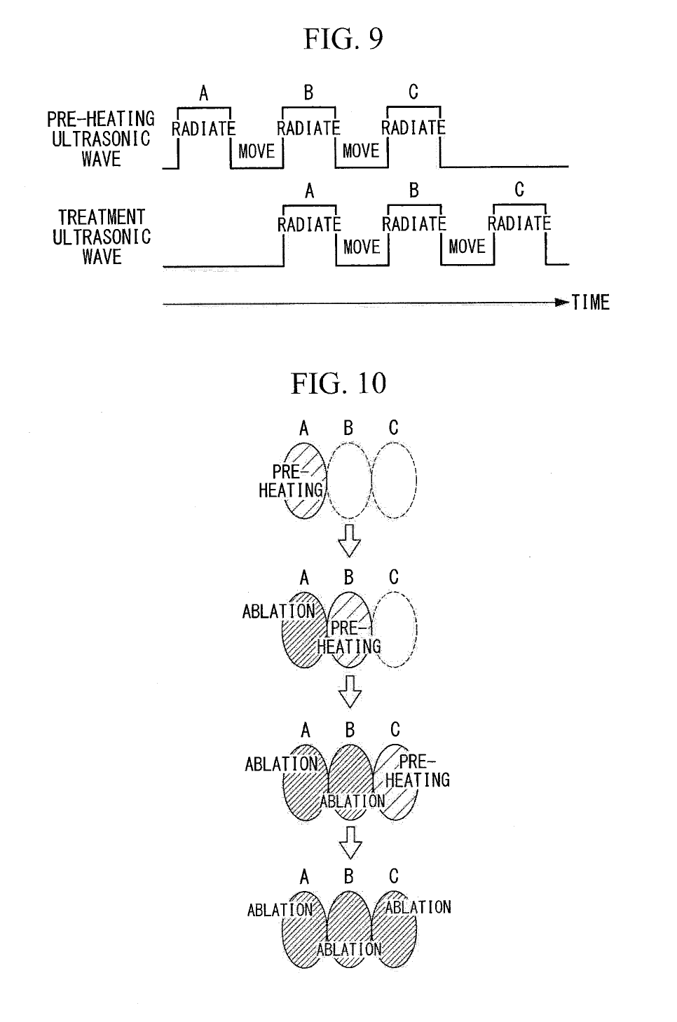

By doing so, it is possible to ablate a large affected portion by dividing it into small regions and by sequentially treating them. The timing of irradiation with the pre-heating ultrasonic waves U2 and the timing of irradiation with the treatment ultrasonic waves U1 may be shifted, as shown in FIGS. 7 and 8, or may be simultaneous, as shown in FIGS. 9 and 10.

In the modifications shown in FIGS. 6 to 10, it is preferable that the pre-heating ultrasonic waves U2 are also focused ultrasonic waves so that the size of the region to be pre-heated by the pre-heating ultrasonic waves U2 is equivalent to the size of the region to be heated, by means of the treatment ultrasonic waves U1, to a temperature that is equal to or greater than the thermal-denaturation temperature. In this way, by limiting the region to be pre-heated, it is possible to prevent regions outside the affected portion from being ablated even if regions outside the affected portion are irradiated with the treatment ultrasonic waves U1.

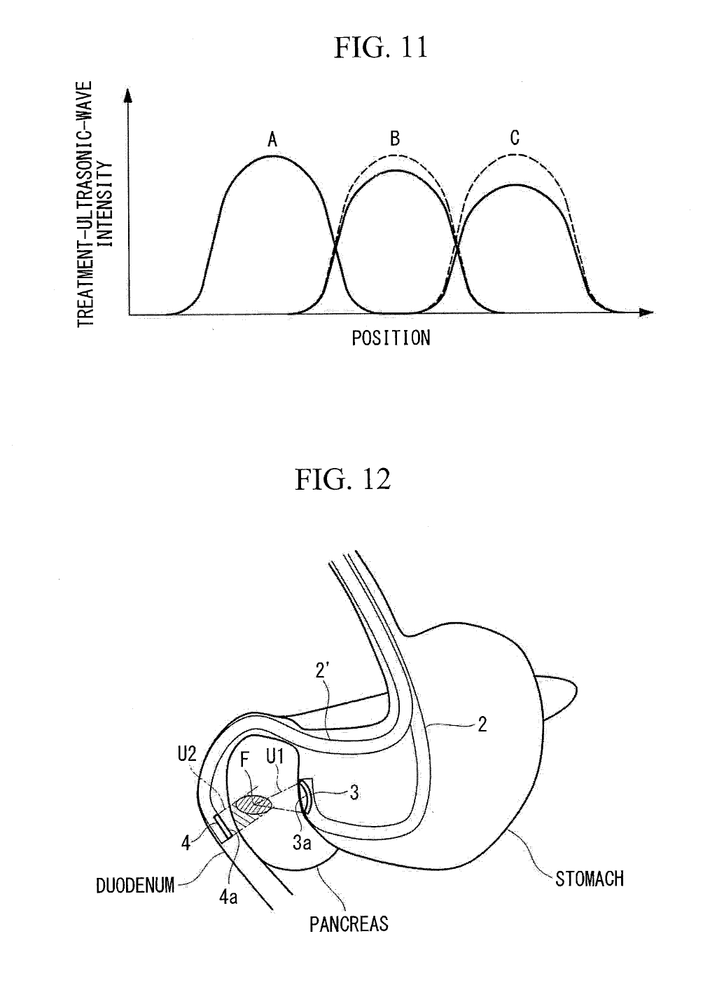

In addition, as shown in FIG. 11, in the modifications shown in FIGS. 6 to 10, the drive control portion (treatment-ultrasonic-wave setting unit) 5 may decrease the intensity of the treatment ultrasonic waves U1 every time the focal point F is moved. When ablating the biological tissue S at the second position and thereafter, because the vicinity of the focal point F is pre-heated to a greater temperature due to heat conduction from the surrounding regions that have already been heated, it is possible to ablate the biological tissue S by using weaker treatment ultrasonic waves U1. In addition to or instead of decreasing the intensity of the treatment ultrasonic waves U1, the irradiation time of the treatment ultrasonic waves U1 may be decreased.

In addition, in this embodiment, although it has been assumed that the treatment-ultrasonic-wave irradiating portion 3 and the pre-heating ultrasonic-wave irradiating portion 4 are provided in the same inserted portion 2, alternatively, as shown in FIG. 12, the treatment-ultrasonic-wave irradiating portion 3 and the pre-heating ultrasonic-wave irradiating portion 4 may be provided in separate inserted portions 2 and 2′. In this case, it is preferable that the treatment-ultrasonic-wave irradiating portion 3 and the pre-heating ultrasonic-wave irradiating portion 4 be disposed facing each other on either side of the affected portion, and that the affected portion is irradiated with the treatment ultrasonic waves U1 and the pre-heating ultrasonic waves U2 from opposite sides from of each other. In the example shown in FIG. 12, the treatment-ultrasonic-wave irradiating portion 3 and the pre-heating ultrasonic-wave irradiating portion 4 are respectively disposed at the stomach and the duodenum, which are positioned on either side of the pancreas, which is the affected portion, and the pancreas are irradiated with the treatment ultrasonic waves U1 and the pre-heating ultrasonic waves U2 from opposite sides from each other.

In addition, in this embodiment, although it is assumed that the affected portion is directly pre-heated by the pre-heating ultrasonic waves U2, alternatively, nearby tissue positioned in the vicinity of the affected portion may be heated and the affected portion may be indirectly pre-heated by means of heat conduction from the heated nearby tissue.

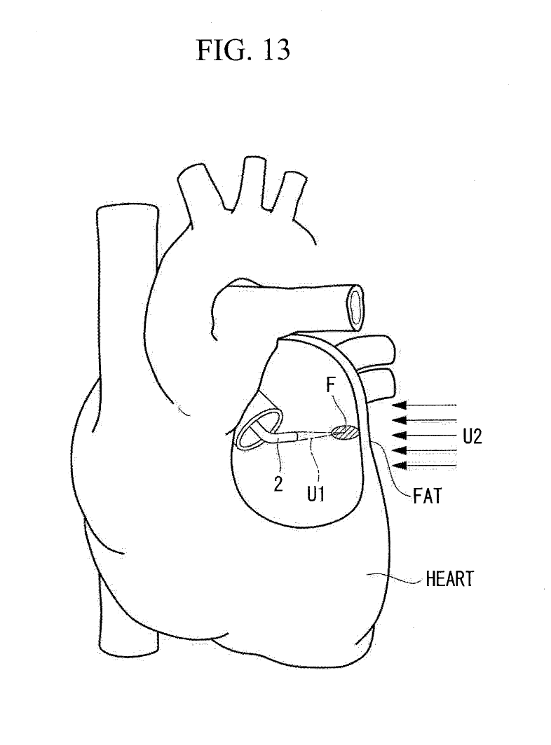

FIG. 13 shows an example in which, in treatment that ablates the heart from inside, a fat that covers a heart surface is irradiated with the pre-heating ultrasonic waves U2 are irradiated, from outside to be heated, and the affected portion is pre-heated by means of heat conduction from the heated fat. Because fat exhibits a greater absorption rate with respect to ultrasonic waves as compared with other types of tissue such as muscle or the like, it is possible to selectively heat fat by using the pre-heating ultrasonic waves U2. It is possible to employ a similar pre-heating method in the treatment of other organs (for example, the liver, stomach, and intestine) in which the surfaces thereof are covered with fat.

In addition, in this embodiment, although it is assumed that the ultrasonic waves U2 are used as energy waves for pre-heating the biological tissue S, alternatively, other types of energy waves, for example, microwaves or laser beams, may be employed.

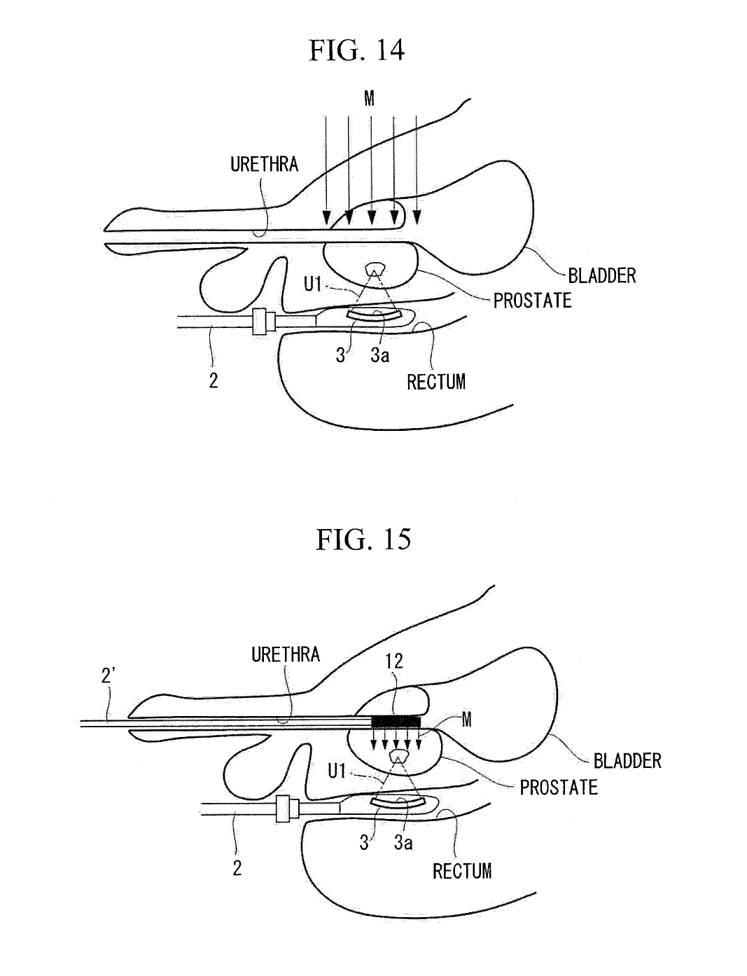

FIG. 14 shows a modification in which microwaves M are emitted instead of the pre-heating ultrasonic waves U2. By radiating microwaves M in a frequency range (for example, 1-20 GHz) in which water has a high absorption rate onto the biological tissue S, it is possible to selectively heat regions in which water is abundantly present, for example, the bladder in which urine is stored and the urethra. Therefore, in treatment of the prostate or the uterus that are positioned in the vicinity of the bladder or the urethra, the bladder or the urethra may be heated by using the microwaves M, and the prostate or the uterus may be indirectly pre-heated by using the bladder or the urethra as a heat source.

Although FIG. 14 shows an external system with which the bladder or the urethra is irradiated with the microwaves M from outside, an internal system with which the affected portion is irradiated with the microwaves M inside the body may be employed.

FIG. 15 shows an example of the internal system. In FIG. 15, the treatment-ultrasonic-wave irradiating portion 3 and a microwave irradiating portion 12 (pre-heating-energy irradiator), which emits the microwaves M, are respectively disposed at the rectum and the urethra, which are positioned on either side of the prostate, which is the affected portion, and the prostate is irradiated with the treatment ultrasonic waves U1 and the microwaves M from opposite sides from each other.

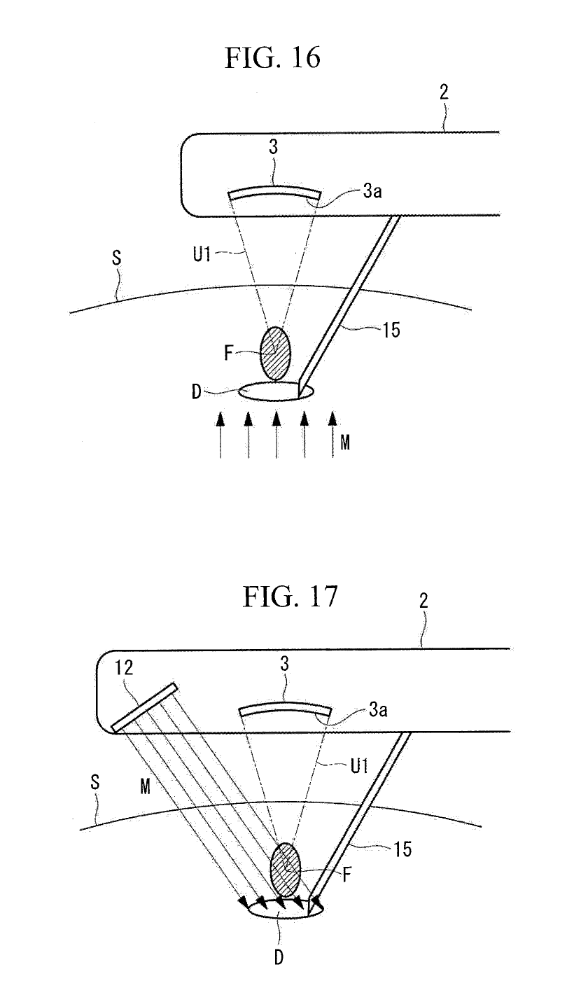

As shown in FIGS. 16 to 18B, in the case in which the microwave irradiating portion 12 is employed, an aqueous solution D such as physiological saline or the like may be injected in the vicinity of the affected portion by using an injection needle 15 that is provided so as to be protruded from a distal-end portion of the inserted portion 2, and the affected portion may be indirectly pre-heated by heating the injected aqueous solution D with the microwaves M. In this case, the aqueous solution D is injected into a position deeper than the affected portion in order to prevent the biological tissue S between the treatment-ultrasonic-wave irradiating portion 3 and the affected portion from being pre-heated.

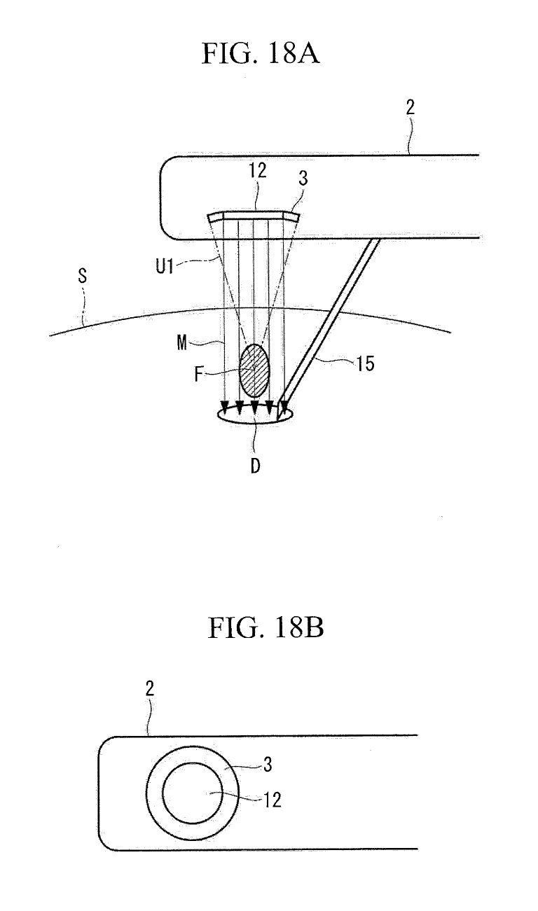

As shown in FIG. 16, the affected portion may be irradiated with the microwaves M from the opposite side of the treatment ultrasonic waves U1. Alternatively, as shown in FIGS. 17 to 18B, the affected portion may be irradiated with the microwaves M from the same side as the treatment ultrasonic waves U1. In FIG. 17, the direction in which the aqueous solution D is irradiated with the microwaves M is different from the direction in which the affected portion is irradiated with the treatment ultrasonic waves U1. In FIGS. 18A and 18B, the direction in which the aqueous solution D is irradiated with the microwaves M is the same as the direction in which the affected portion is irradiated with the treatment ultrasonic waves U1. In the case in which the absorption of the microwaves M at the affected portion is sufficiently small as compared with the absorption of the microwaves M at the injected aqueous solution D, the surface temperature of the biological tissue S is not increased relative to the heating of the aqueous solution D during irradiation with the microwaves M. Therefore, as shown in FIGS. 18A and 18B, an annular emitting surface of the treatment-ultrasonic-wave irradiating portion 3 and a circular emitting surface of the microwave irradiating portion 12 may be coaxially disposed so that the treatment ultrasonic waves U1 and the microwaves M are coaxially emitted.

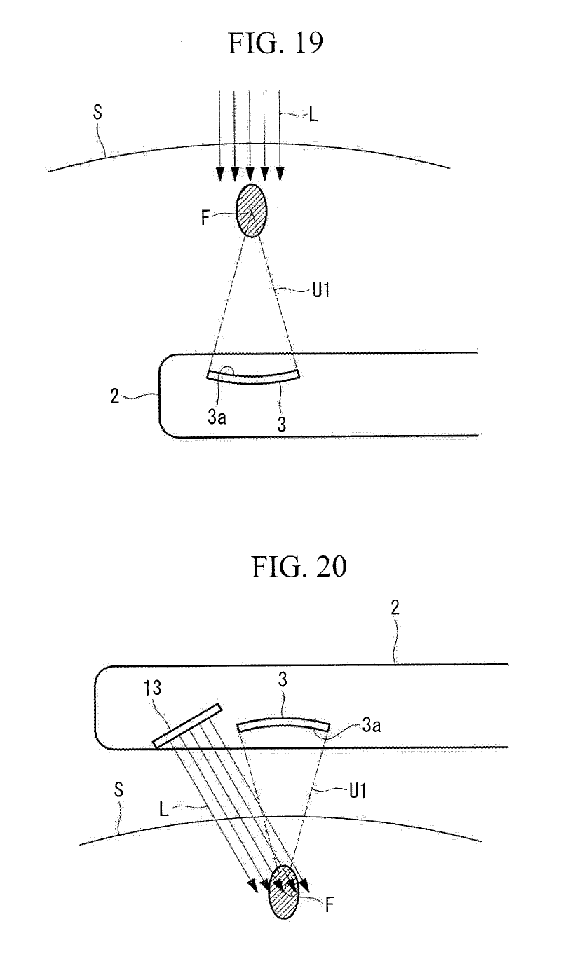

FIGS. 19 and 20 show modifications that are provided with, instead of the pre-heating ultrasonic-wave irradiating portion 4, a laser-beam irradiating portion 13 (pre-heating-energy irradiator) that irradiates the biological tissue S with laser beams L. By irradiating the biological tissue S with laser beams L in a wavelength region in which a specific component is contained in the biological tissue S exhibits a high absorption rate, it is possible to selectively heat a specific region of the biological tissue S.

Although laser beams in a wavelength region at or above 1100 nm are absorbed approximately equally by vascular tissue and tissue that does not contain blood vessels, because these laser beams are strongly absorbed by water molecules in the biological tissue S, it is possible to selectively heat regions in which water molecules are abundant.

Because laser beams L in a wavelength region below 1100 nm are more strongly absorbed by vascular tissue than tissue that does not contain blood vessels, it is possible to selectively heat the vascular tissue. For example, in the case in which laser beams L in a wavelength region near 400 nm, in which red blood cells exhibit a high absorption rate, are employed, the blood vessels are selectively heated. In particular, in the case in which laser beams L at about 900 nm, which is the wavelength at which oxyhemoglobin exhibits an absorption peak, are employed, blood vessels containing abundant oxygen, such as new blood vessels or the like, are selectively heated. Therefore, it is possible to selectively pre-heat, by using the laser beams L, a tumor in which capillaries and new blood vessels are abundantly present and blood flow is moderate.

In the case in which an increase in the temperature in blood vessels due to the irradiation with the laser beams L is sufficiently greater than an increase in the temperature in other tissue in the affected portion, the laser-beam irradiating portion 13 may be disposed in a similar manner as the microwave irradiating portion 12 shown in FIGS. 18A and 18B, and the affected portion may be irradiated with the treatment-ultrasonic waves U1 and the laser beams L from the same direction.

When heating blood vessels in which blood flow is rapid, the blood vessels may be irradiated with the laser beams L in a state in which the blood flow is stopped by means of pressure or the like.

The laser beams L may be standing waves or may be high-frequency pulses. In the case in which high-frequency pulses, which possess greater energy as compared with the standing waves, are employed, it is possible to more efficiently pre-heat the biological tissue S.

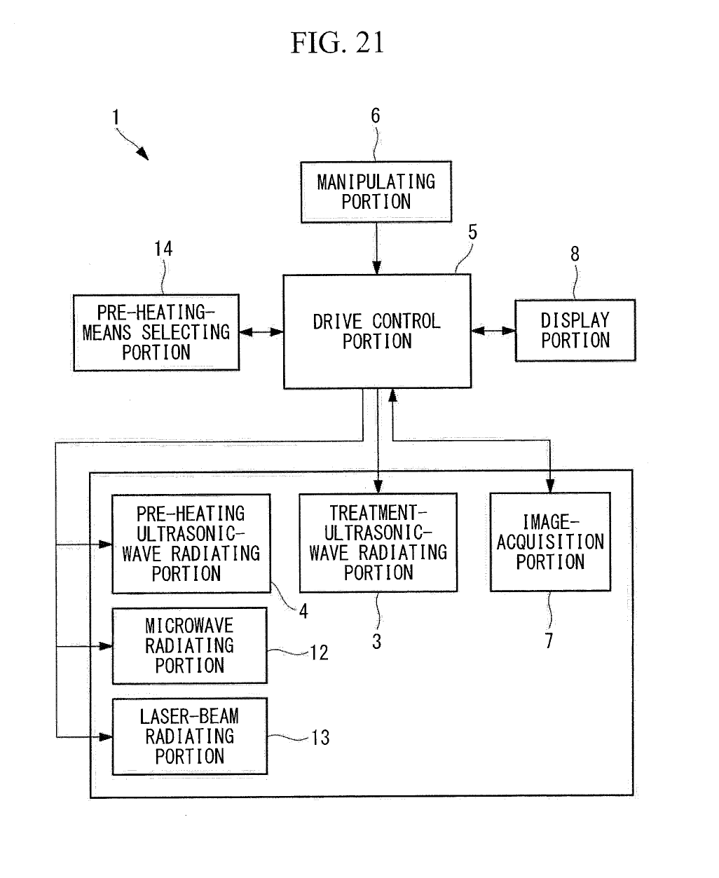

In addition, this embodiment may be provided with the multiple types of pre-heating-energy irradiating portions 4, 12, and 13, described above, and, additionally, a pre-heating-means selecting portion 14 (pre-heating-means selector) that selects an appropriate type of pre-heating-energy irradiating portion in accordance with the treatment conditions and recommends it to the user. Although FIG. 21 shows, as an example, a configuration in which the pre-heating-energy irradiating portions 4, 12, and 13 and the treatment-ultrasonic-wave irradiating portion 3 are provided at the distal-end portion of the same inserted portion 2, the pre-heating-energy irradiating portions 4, 12, and 13 may be provided in an inserted portion that is separate from the inserted portion in which the treatment-ultrasonic-wave irradiating portion 3 is provided, and an external system that irradiates energy waves from outside may be employed.

The pre-heating-means selecting portion 14 selects the type of pre-heating-energy irradiating portions 4, 12, and 13 on the basis of the treatment conditions input by the user via the manipulating portion (input unit) 6. The treatment conditions are, for example, a disease and an organ to be treated, the thickness of that organ, and so forth. For example, in the case in which the disease to be treated is cancer, the pre-heating-means selecting portion 14 recommends the laser-beam irradiating portion 13 that outputs laser beams L having an output wavelength of 660 nm, and, in the case in which the organ to be treated is the prostate, the pre-heating-means selecting portion 14 recommends the microwave irradiating portion 12. By doing so, it is possible to support the user in selecting the optimum pre-heating-energy irradiating portion 4, 12, or 13 for the treatment.

As a result, the following aspect is read by the above described embodiment of the present invention.

An aspect of the present invention provides an ultrasonic treatment apparatus comprising: a treatment-ultrasonic-wave irradiator that is disposed facing a surface of a biological tissue and that is configured to irradiate the biological tissue with focused ultrasonic waves, thus heating a vicinity of a focal point of the focused ultrasonic waves positioned at a deep portion in the biological tissue to a temperature that is equal to or greater than a thermal-denaturation temperature of the biological tissue; and a pre-heating-energy irradiator that is configured to irradiate the biological tissue with energy waves, thus heating the vicinity of the focal point to a temperature that is less than the thermal-denaturation temperature, wherein the pre-heating-energy irradiator is configured to irradiate the biological tissue with the energy waves from a direction that is different from the direction in which the treatment-ultrasonic-wave irradiator irradiates with the focused ultrasonic waves.

With the above-described aspect, the treatment-ultrasonic-wave irradiator is disposed facing the biological tissue so that the focal point of the focused ultrasonic waves is aligned with the affected portion positioned in a deep portion of the biological tissue, and, when the biological tissue is irradiated with the focused ultrasonic waves from the treatment-ultrasonic-wave irradiator, the ultrasonic waves are focused at the affected portion, and thus, the affected portion is locally heated and ablated. Here, by pre-heating the vicinity of the affected portion by irradiating the biological tissue with the energy waves from the pre-heating-energy irradiator before irradiating with the focused ultrasonic waves, it is possible to decrease the energy and the irradiation time of the focused ultrasonic waves that are necessary to ablate the affected portion as compared with the case in which the vicinity of the affected portion is not pre-heated.

In this case, the biological tissue positioned between the treatment-ultrasonic-wave irradiator and the focal point is not pre-heated by the energy waves. Therefore, when the biological tissue is irradiated with the focused ultrasonic waves until the affected portion is ablated after pre-heating, in the portion between the treatment-ultrasonic-wave irradiator and the focal point, in particular at the surface of the biological tissue, the focused ultrasonic waves is prevented from being made heated the biological tissue to a temperature that is equal to or greater than the thermal-denaturation temperature. By doing so, it is possible to prevent the surface of and inside the biological tissue in the pathway through which the biological tissue is irradiated with the ultrasonic waves from being heated, and it is possible to selectively ablate only the affected portion.

In the above-described aspect, the pre-heating-energy irradiator may irradiate the biological tissue with the energy waves from a direction that is different from the direction in which the focused ultrasonic waves are irradiated by the treatment-ultrasonic-wave irradiator.

By doing so, because the propagation pathway of the energy waves and the propagation pathway of the focused ultrasonic waves are different, it is possible to more reliably prevent the same area of the biological tissue from being heated by both the energy waves and the focused ultrasonic waves.

The above-described aspect, may further comprise: a pre-heat-temperature measuring instrument that measures a temperature in the vicinity of the focal point heated by the pre-heating-energy irradiator; and a treatment-ultrasonic-wave setting unit that sets, on the basis of the temperature measured by the pre-heat-temperature measuring instrument, at least one of an intensity and a irradiation time of the focused ultrasonic waves to irradiate the biological tissue from the treatment-ultrasonic-wave irradiator.

By doing so, it is possible to reliably ablate the affected portion by the affected portion is adequately irradiated with the right amount of the ultrasonic waves in accordance with the temperature of the affected portion that has been pre-heated by the irradiation with the energy waves.

In the above-described aspect, the pre-heat-temperature measuring instrument may be provided with a temperature sensor that takes an actual measurement of the temperature in the vicinity of the focal point.

By doing so, it is possible to obtain a more accurate temperature of the affected portion.

In the above-described aspect, the pre-heat-temperature measuring instrument may calculate the temperature in the vicinity of the focal point on the basis of irradiation conditions of the energy waves by the pre-heating-energy irradiator.

By doing so, because equipment such as a sensor or the like is not necessary, it is possible to simplify the apparatus configuration.

The above-described aspect, may further comprise: a treatment-region moving mechanism that is configured to move the focal point of the focused ultrasonic waves radiated from the treatment-ultrasonic-wave irradiator which irradiate the biological tissue; a pre-heating-region moving mechanism that is configured to move a irradiation region of the energy waves from the pre-heating irradiating-energy irradiator which irradiates the biological tissue; and a controller that controls the treatment-ultrasonic-wave irradiator, the energy irradiator, the treatment-region moving mechanism, and the pre-heating-region moving mechanism so that heating of the irradiation region by means of the energy waves and heating of the irradiating region that has been heated by the energy wave in an immediately preceding step by means of the focused ultrasonic waves are executed in an alternating manner while changing the position of the irradiation region and the focal point.

By doing so, it is possible to efficiently ablate a large area in the biological tissue.

In the above-described aspect, the energy waves may be ultrasonic waves.

By doing so, it is possible to pre-heat the biological tissue by converting the vibrational energy possessed by the ultrasonic waves to the thermal energy in the biological tissue. In particular, because fat exhibits a greater absorption rate with respect to ultrasonic waves as compared with other types of tissue, it is possible to selectively pre-heat fat by using the ultrasonic waves.

In the above-described aspect, the energy waves may be microwaves.

By doing so, it is possible to pre-heat the biological tissue by converting the electromagnetic energy possessed by the microwaves to the thermal energy in the biological tissue. In particular, the water molecules exhibit a high absorption rate with respect to microwaves in the frequency range of 1-20 GHz. Therefore, it is possible to efficiently and selectively pre-heat a region in which water molecules are abundantly present by using the microwaves in the above-described frequency range.

In the above-described aspect, the energy waves may be laser beams.

By doing so, it is possible to pre-heat the biological tissue by converting the light energy possessed by the laser beams to the thermal energy in the biological tissue. Vascular tissue exhibits a greater energy absorption than tissue that does not contain blood vessels, with respect to light in a wavelength region below 1100 nm, and thus, this light tends to be converted to thermal energy in vascular tissue. In particular, red blood cells exhibit a high absorption rate with respect to light in a wavelength region of near 400 nm, deoxyhemoglobin exhibits a high absorption rate with respect to light in a wavelength region above and below 660 nm, and oxyhemoglobin exhibits a high absorption rate with respect to light in a wavelength region at or above 900 nm. Therefore, it is possible to selectively pre-heat blood vessels in the above-described wavelength regions by using laser beams in the above-described wavelength regions.

The above-described aspect, may further comprise: a plurality of types of the pre-heating-energy irradiator, which output mutually different types of the energy waves; an input unit with which a user inputs a treatment condition; and a pre-heating-means selector that is configured to select the type of the pre-heating-energy irradiator to be used in treatment in accordance with the treatment condition input via the input unit.

By doing so, it is possible to support the user in appropriately selecting the type of the pre-heating-energy irradiator.

### REFERENCE SIGNS LIST

1 ultrasonic treatment apparatus
2 inserted portion
3 treatment-ultrasonic-wave irradiator
4 pre-heating ultrasonic-wave irradiating portion (pre-heating-energy irradiator)
5 drive control portion (controller, treatment-ultrasonic-wave setting unit)
6 manipulating portion (input unit)
7 image-acquisition portion
8 display portion
9 pre-heat-temperature measuring portion (pre-heat-temperature measuring instrument)
10 treatment-region moving mechanism
11 pre-heating-region moving mechanism
12 microwave irradiating portion (pre-heating-energy irradiator)
13 laser-beam irradiating portion (pre-heating-energy irradiator)
14 pre-heating-means selecting portion (pre-heating-means selector)
15 injection needle
U1 treatment ultrasonic wave (focused ultrasonic wave)
U2 pre-heating ultrasonic wave (pre-heating energy wave)
M microwaves (pre-heating energy wave)
L laser beam (pre-heating energy wave)

1. An ultrasonic treatment apparatus comprising:
a treatment-ultrasonic-wave irradiator that is disposed facing a surface of biological tissue and that is configured to irradiate the biological tissue with focused ultrasonic waves, thus heating a vicinity of a focal point of the focused ultrasonic waves positioned at a deep portion in the biological tissue to a temperature that is equal to or greater than a thermal-denaturation temperature of the biological tissue; and
a pre-heating-energy irradiator that is configured to irradiate the biological tissue with energy waves, thus heating the vicinity of the focal point to a temperature that is less than the thermal-denaturation temperature,
wherein the pre-heating-energy irradiator is configured to irradiate the biological tissue with the energy waves from a direction that is different from the direction in which the treatment-ultrasonic-wave irradiator irradiates with the focused ultrasonic waves.

2. An ultrasonic treatment apparatus according to claim 1, further comprising:
a pre-heat-temperature measuring instrument that is configured to measure a temperature in the vicinity of the focal point heated by the pre-heating-energy irradiator; and
a treatment-ultrasonic-wave setting unit that is configured to set, on the basis of the temperature measured by the pre-heat-temperature measuring instrument, at least one of an intensity and a irradiation time of the focused ultrasonic waves to irradiate the biological tissue from the treatment-ultrasonic-wave irradiator.

3. An ultrasonic treatment apparatus according to claim 2, wherein the pre-heat-temperature measuring instrument is provided with a temperature sensor that takes an actual measurement of the temperature in the vicinity of the focal point.

4. An ultrasonic treatment apparatus according to claim 2, wherein the pre-heat-temperature measuring instrument is configured to calculate the temperature in the vicinity of the focal point on the basis of irradiation conditions of the energy waves by the pre-heating-energy irradiator.

5. An ultrasonic treatment apparatus according to claim 1, further comprising:
a treatment-region moving mechanism that is configured to move the focal point of the focused ultrasonic waves from the treatment-ultrasonic-wave irradiator which irradiates the biological tissue;
a pre-heating-region moving mechanism that is configured to move a irradiation region of the energy waves from the pre-heating-energy irradiator which irradiates the biological tissue; and
a controller that is configured to control the treatment-ultrasonic-wave irradiator, the energy irradiator, the treatment-region moving mechanism, and the pre-heating-region moving mechanism so that heating of the irradiation region by means of the energy waves and heating of the irradiation region that has been heated by the energy waves in an immediately preceding step by means of the focused ultrasonic waves are executed in an alternating manner while changing the position of the irradiation region and the focal point.

6. An ultrasonic treatment apparatus according to claim 1, wherein the energy waves are ultrasonic waves.

7. An ultrasonic treatment apparatus according to claim 1, wherein the energy waves are microwaves.

8. An ultrasonic treatment apparatus according to claim 1, wherein the energy waves are laser beams.

9. An ultrasonic treatment apparatus according to claim 1, further comprising:
a plurality of types of the pre-heating-energy irradiator, which output mutually different types of the energy waves;
an input unit with which a user inputs a treatment condition; and
a pre-heating-means selector that is configured to select the type of the pre-heating-energy irradiator to be used in treatment in accordance with the treatment condition input via the input unit.

10. An ultrasonic treatment apparatus according to claim 1, wherein the treatment-ultrasonic-wave irradiator and the pre-heating-energy irradiator are disposed to be inclined with respect to each other so that a sound axis of the focused ultrasonic waves and an irradiation axis of the energy waves intersect with each other at the focal point of the focused ultrasonic waves.

11. An ultrasonic treatment apparatus according to claim 5, wherein the controller is configured to control a timing of irradiating with the energy waves and a timing of irradiating with the focused ultrasonic waves being simultaneous.

12. An ultrasonic treatment apparatus according to claim 5, wherein the controller is configured to control a timing of irradiating with the energy waves and a timing of irradiating with the focused ultrasonic waves being shifted.
